(19) United States
(12) Patent Application Publication
(10) Pub. No.: US 2026/0177518 A1
(43) Pub. Date: Jun. 25, 2026

(54) WOOD MICROFLUIDIC ENGINEERING: DEVELOPMENT OF HIGHLY STABLE POLYMER NANOCOMPOSITE ELECTROCHEMICAL NITRATE SENSOR

(71) Applicant: UNIVERSITY OF MARYLAND, BALTIMORE COUNTY, Baltimore, MD (US)

(72) Inventors: Govind Rao, Baltimore, MD (US); Sanjeev Kumar, Baltimore, MD (US); Venkatesh Srinivasan, Baltimore, MD (US); Preety Ahuja, Baltimore, MD (US); Revati Kadolkar, Baltimore, MD (US)

(21) Appl. No.: 19/541,735

(22) Filed: Feb. 17, 2026

Related U.S. Application Data
(63) Continuation of application No. PCT/US2024/041166, filed on Aug. 7, 2024.
(60) Provisional application No. 63/520,532, filed on Aug. 18, 2023.

(51) Int. Cl. G01N 27/30; G01N 27/416; G01N 33/18

(52) U.S. Cl. CPC G01N 27/308; G01N 27/4166; G01N 33/182

(57) ABSTRACT

A biosustainable wood-based microfluidic cassette having an impedimetric nitrate sensor for nitrate monitoring of water samples, wherein the nitrate sensor employs a nanocomposite having nitrate-binding metal ion complexes incorporated in and/or on polyaniline/carbon nanostructure. The nitrate sensor demonstrates good specificity to nitrate, reproducibility, a wide temperature range operability, and measurement equivalency to commercial nitrate sensors.

### CROSS REFERENCE TO RELATED APPLICATIONS

This application is a continuation application claiming priority to International Patent Application No. PCT/US2024/041166 filed on 7 Aug. 2024, which claims priority to U.S. Provisional Patent Application No. 63/520,532 filed on Aug. 18, 2023 in the name of Govind RAO et al. entitled “WOOD MICROFLUIDIC ENGINEERING: DEVELOPMENT OF HIGHLY STABLE POLYMER NANOCOMPOSITE ELECTROCHEMICAL NITRATE SENSOR,” both of which are hereby incorporated by reference herein in their entirety.

### STATEMENT OF FEDERALLY SPONSORED RESEARCH

This invention was made with government support under Grant No. 2012340 awarded by the National Science Foundation. The government has certain rights in the invention.

### FIELD OF THE INVENTION

This invention relates to polymer-carbon nanocomposites used for nitrate detection in ecofriendly wood microfluidic cassettes.

### BACKGROUND OF THE INVENTION

The availability of high-quality drinking water is the 6^{th} out of 17 Sustainable Development Goals (SDG) global indicators listed by the United Nations which reiterates the importance of continuous monitoring of water quality [Obaideen, 2022]. Continuous monitoring of water quality is increasingly becoming a necessity due to the constant release of toxic effluents to surface water and groundwater. Nitrate (NO₃⁻) is one such contaminant that is released into the environment through synthetic and agricultural fertilizers, livestock manure discharge, atmospheric septic system discharge, and sanitary sewer leaks. This has serious implications on both aquatic and human lives. High nitrate contamination leads to algal blooms in lakes and rivers, resulting in oxygen-depleted zones causing hypoxia in aquatic life [Banerjee, 2023]. In humans, nitrate can lead to gastric cancer as nitrites form carcinogenic N-nitrosamines upon interaction with amines/amides in the stomach [Mirvish, 1995]. Prolonged nitrate accumulation may result in birth defects, spontaneous abortion, intrauterine growth restriction, and methemoglobinemia, also called the blue baby syndrome, which results from binding of nitrate to hemoglobin, thereby causing depletion of oxygen in body tissues [Fossen Johnson, 2019].

To address these health concerns, the World Health Organization has set a maximum allowable concentration of 10 mg/L or 10 ppm of nitrate-nitrogen in drinking water. Nitrate contamination is presently on the rise in several regions of Asia, Africa, and Europe [Abascal, 2021]. In the United States, around 86% of the population relies on public water systems (PWS) for their water supply. Studies reveal that a significant number of regions are receiving water from systems that contain elevated levels of nitrate due to inadequate monitoring of groundwater nitrate [Pennino, 2017]. Consequently, a considerable portion of the population is exposed to water contaminated with nitrate.

However, despite the increasing demand for real-time on-site monitoring of nitrate levels, there is a lack of low-cost and sensitive real-time commercial sensors for monitoring nitrate contamination through frequent testing. One of the reasons for this unavailability is the inherent complexities in direct and indirect measurement techniques of nitrate namely electrophoresis and polarography [Glazier, 1998]. Typically, nitrate (NO₃⁻) is first reduced to nitrite ions (NO₂⁻) and then quantified using spectrophotometry [Johnson, 2022]. This approach has multiple steps, large interferences and a limited detection range [Badea, 2001]. Various alternative laboratory techniques used for nitrate detection, such as colorimetry [Charbaji, 2021; Le Goff, 2002], molecular imprinting [Essousi, 2019], ion chromatography [Butt, 2001; Wang, N., 2012], and field-effect transistor technology [Chen, 2018] involve labor-intensive sampling, require advanced instrumentation, and are time-consuming. Commercially used sensors like YSI are highly selective for nitrate but need frequent calibration [Snyder, 2018]. Furthermore, disadvantageously, these prior art techniques of sensing nitrate are frequently accompanied by the unintended release of other toxins into the environment. Moreover, the disposal of such sensors contributes to the environmental burden, exacerbating the impact on ecosystems [Cogan, 2015].

One solution to this problem is to limit the quantity of reagents used for sensing and use a microfluidic set up. The development of microfluidics has led to the miniaturization of analytical devices, which has significant implications for automated monitoring platforms. Advantages of using microfluidics include lower response time, improved selectivity, and automation of laboratory experiments into a portable device.

There have been several optical, electrochemical and paper-based microfluidic nitrate sensors developed in the prior art [Li, 2023]. For example, a PMMA (Poly methyl methacrylate) microfluidic chip for nitrate detection using the Griess method has been developed [Khanfar, 2017]. However, reagent-based colorimetric sensors are temperature sensitive and some use toxic chemicals such as cadmium [Gal, 2004]. Charbaji et al. developed a paper based microfluidic device made of a composite containing cotton fibers and zinc microparticle with a limit of quantification of 1.765 ppm of nitrate but disadvantageously the developed sensor has a limited shelf-life and uses large quantities of reagents [Charbaji, 2021].

While polymer-based microfluidic chips offer convenience in processing and handling, they raise concerns regarding their eco-friendliness. Accordingly, there continues to be a need for environmentally friendly microfluidic devices that can be used to detect nitrate. Towards that end, a microfluidic device using wood-based materials for rapid nitrate detection using electrochemical methods is disclosed. Wood microfluidics not only demonstrate sustainability and environmental compatibility but also exhibit comparable process-handling and usability, relative to conventional plastic microfluidic cassettes. The nitrate sensor described herein merges the advantages of microfluidic systems and electrochemical detection while championing sustainability by utilizing environmentally friendly materials for both the nitrate sensor and the sample handling components.

### SUMMARY OF THE INVENTION

In some aspects, a nanocomposite comprising nitrate-binding metal ion complexes incorporated in and/or on a polyaniline/carbon (Pani/C) nanostructure is described.

In other aspects, a graphite-containing sensor electrode comprising a nanocomposite adhered thereto is described herein, wherein the nanocomposite comprises comprising nitrate-binding metal ion complexes incorporated in and/or on a polyaniline/carbon (Pani/C) nanostructure.

In some other aspects, an apparatus for detecting nitrate (NO₃⁻) ions in a water sample is described, said apparatus comprising:
a microfluidic cassette comprising:
   a microwell having an inlet and an outlet for introduction of the water sample to the microwell and removal of same therefrom;
   a first bore that accommodates a working electrode such that the working electrode is in contact with the contents of the microwell; and
   a second bore that accommodates a reference electrode and a counter electrode, wherein the reference electrode and the counter electrode are in contact with the contents of the microwell and in proximity to the working electrode.
      In some embodiments, the working electrode comprises a graphite-containing sensor electrode comprising a nanocomposite adhered thereto, wherein the nanocomposite comprises comprising nitrate-binding metal ion complexes incorporated in and/or on a polyaniline/carbon (Pani/C) nanostructure.

In some other aspects, a method of measuring a concentration of nitrate ions in a water sample is described, said method comprising:
introducing a liquid buffer to the inlet of an apparatus for detecting nitrate (NO₃⁻) ions in a water sample;
introducing the water sample to the apparatus by dipping the cassette in water or by introducing the water sample to the inlet of the cassette; and
measuring the impedance or electrical current using an electrochemical analyzer and converting same to nitrate concentration.
   In some embodiments, the apparatus comprises a microfluidic cassette comprising: a microwell having an inlet and an outlet for introduction of the water sample to the microwell and removal of same therefrom; a first bore that accommodates a working electrode such that the working electrode is in contact with the contents of the microwell; and a second bore that accommodates a reference electrode and a counter electrode, wherein the reference electrode and the counter electrode are in contact with the contents of the microwell and in proximity to the working electrode. In some embodiments, the working electrode comprises a graphite-containing sensor electrode comprising a nanocomposite adhered thereto, wherein the nanocomposite comprises comprising nitrate-binding metal ion complexes incorporated in and/or on a polyaniline/carbon (Pani/C) nanostructure.

Other aspects and advantages will be more fully apparent from the ensuing disclosure and appended claims.

### BRIEF DESCRIPTION OF THE FIGURES

FIG. 1 illustrates schematic representation of acrylate-based microfluidic sensor cell assembly (10) comprising a working electrode (20), comprising a Ni@Pani/C nanocomposite, on graphite (18), and an electrode (22) comprising a reference electrode (5) and a counter electrode (4).

FIG. 2 illustrates individual layers of the microfluidic sensor cell of FIG. 1 both as filled layers (left) and wire frame layers (right).

FIG. 3A shows the artistic rendering of the nanocomposite Ni@Pani/C coated on the graphite sensor electrode.

FIG. 3B is a SEM micrograph of Ni@Pani/C for surface morphology analysis.

FIG. 3C is a TEM) image of Ni@Pani/C.

FIG. 4 illustrates schematic representation of wood-based microfluidic sensor cell assembly.

FIG. 5A illustrates an exploded view of the wood-based microfluidic sensor cell showing individual layers.

FIG. 6A is an illustration of a possible structure of the prepared nanocomposite (Ni@Pani/C).

FIG. 6B shows the elemental phase analysis performed on powdered samples of Ni@Pani/C using X-ray diffractometer (XRD).

FIG. 6C shows the Raman spectra of powdered samples of Ni@Pani/C.

FIG. 6D shows the FTIR spectra of powdered samples of Ni@Pani/C.

FIG. 7A is a EIS Nyquist plot of bare graphite electrode, Pani/C and Ni@Pani/C nanocomposite in 0.1 M PBS (pH 7.0) containing 5 mM [K₃Fe(CN)₆] and 0.1 M KCl. Inset: 3D schematic representation of the 75 mL conventional three electrode cell assembly.

FIG. 7B is a plot of |Z| vs. ω^{-1/2} from impedance data for Pani/C and Ni@Pani/C nanocomposite

FIG. 7C shows CV plots of Ni@Pani/C at different scan rates (5 mV/s to 200 mV/s). Redox peak currents were monitored with increase in scan rates

FIG. 7D shows the variation of redox peak currents versus scan rate (5 mV/s to 200 mV/s).

FIG. 7E shows CV plots of Ni@Pani/C nanocomposite at 50 mV/s and different pHs of the electrolyte: 4.5, 6.0 and 7.0.

FIG. 7F shows CV plots of Ni@Pani/C nanocomposite at 50 mV/s in different electrolytes compositions.

FIG. 8A is a CV of Ni@Pani/C sensing electrode at 50 mV/s in the absence and presence of 5 ppm nitrate at electrolyte pH 4.5, 6.0 and 7.0, Outset: Plot showing change in current with pH.

FIG. 8B is a calibration curve of change in charge transfer resistance (ΔR_{CT}) in the Nyquist plot with increasing concentration of nitrate from 0-50 ppm for Ni@Pani/C sensor.

FIG. 8C is an EIS Nyquist plots of Ni@Pani/C sensor upon successive injection of different concentrations of nitrate (0-20 ppm) in N₂ saturated 0.1 M PBS and pH 6.0 electrolyte at −0.45 V.

FIG. 9A. (Left) Schematic representation of the microfluidic cassette, as shown in more detail in FIGS. 4 and 5. (Right) PMMA-based microfluidic electrochemical sensor setup with Ni@Pani/C nanocomposite coated on 1 cm² graphite plate, screen printed conducting carbon and Ag/AgCl working, counter and reference electrodes, respectively. Description shows dimensions and weight of the microfluidic cassette.

FIG. 9B shows CV plots of the Ni@Pani/C sensor at 50 mV/s in the absence and presence of 5 ppm nitrate.

FIG. 9C shows EIS Nyquist plots of the Ni@Pani/C sensor upon successive injection of different concentrations of nitrate (0-10 ppm) in N₂ saturated 0.1 M PBS and pH 6.0 electrolyte at −0.45 V.

FIG. 9D is a calibration curve with increasing molar concentration of nitrate from 0.6-50 ppm obtained from the Ni@Pani/C sensor.

FIG. 9E shows amperometry sensing with repeated additions of 0.4 ppm and 1 ppm of nitrate in 0.1M PBS and pH 6.0 electrolyte.

FIG. 10A shows the comparative performance of the Ni@Pani/C electrochemical sensor described herein versus commercial SUNA V2 with stream samples from Gwynns Run, Alexander Avenue and Dead Run watershed, all within the Gwynns Falls watershed in Maryland, U.S.A.

FIG. 10B shows Ni@Pani/C sensor's EIS response for 4 ppm nitrate and 4 ppm of interfering ions: sulphate, bicarbonate, acetate, and ammonium.

FIG. 10C shows the Ni@Pani/C sensor performance at different temperatures maintaining a fixed 2 ppm nitrate concentration.

FIG. 10D shows the nitrate sensing response of the Ni@Pani/C sensor with long term storage (26 days). Sensing performed in pH 6.0 at room temperature.

FIG. 11A shows the electropolymerization of the Ni@Pani/C sensor over 200 CV cycles at pH 0.5±0.1.

FIG. 11B shows corrosion current measurements by Tafel plot in a basic medium (pH 14.04±0.3).

FIG. 12A shows CV plots of Ni@Pani/C WMC sensor at 50 mV/s in the absence and presence of 5 ppm nitrate.

FIG. 12B shows a Langmuir plot of Ni@Pani/C WMC sensor for a nitrate concentration of 0-60 ppm, comparing the experimentally determined values relative to the Langmuir model.

FIG. 12C shows EIS Nyquist plots of Ni@Pani/C WMC sensor upon successive injection of different concentrations of nitrate (0-10 ppm) in N₂ saturated 0.1 M PBS and pH 6.0 electrolyte at −0.45 V.

FIG. 12D is a calibration curve with increasing molar concentration of nitrate from 0.6-60 ppm obtained from the Ni@Pani/C WMC sensor.

FIG. 13A shows WMC sensor's EIS response for 4 ppm nitrate and 4 ppm of interfering ions: sulphate, bicarbonate, acetate, and ammonium.

FIG. 13B shows the long-term WMC stability performance for 12 months and various sampling over 100 cycles.

FIG. 13C shows the effect of temperature on the structural integrity of the WMC range from 4° C. to 60° C.

FIG. 13D shows the comparative performance of the WMC electrochemical sensor described herein versus commercial SUNA V2 with stream samples from Gwynns Run, Alexander Avenue and Dead Run watershed, all within the Gwynns Falls watershed in Maryland, U.S.A.

FIG. 14 shows the CV plots demonstrating comparative redox peaks of Pani/C(20), Pani/C(65), and Ni@Pani/C(65).

The features and advantages of the invention are more fully illustrated by the following non-limiting example, wherein all components are used in a particular form to demonstrate the usability and practice.

### DETAILED DESCRIPTION, AND PREFERRED EMBODIMENTS THEREOF

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, +100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Electrochemical nitrate sensing that relies on oxidation reactions of such materials and primarily composed of copper or silver, usually encounters interference from other oxidizable species. One effective approach to enhance the selectivity of metallic nanoparticles towards nitrate involves the use of modified functional materials aimed at minimizing operational electrode potential. Among such functional materials, conducting polymers like Pani have shown promise for electrochemical sensing due to their biocompatibility, low toxicity, facile synthesis, cost-effective large-scale production, electro-active redox potentials, high capacity to stabilize enzymes, and the ability to anchor metal nanoparticles with the aid of amino groups. Pani-carbon composites, wherein the carbon is graphene or CNT, are known in the prior art due to carbon's versatility as an electrode modifier and the ability to facilitate an enhanced electron transfer between the electroactive analytes and electrode surfaces [Bagheri, 2017]. Pani/CNT electrodes have been explored for the electrochemical detection of nitrate in tap water, where the oxygen functional groups in CNT adsorb radical cations in the emeraldine salt (ES) form of Pani, thereby making it thermodynamically more favorable for nitrate complex formation [Kosa, 2022]. However, the sensing performance of Pani still depends on the synthesis method, morphology, and loading amount. While graphene and CNT-based nanocomposites offer high electrical conductivity, their practical applications in electrochemical sensors can be hindered by issues such as production scalability and functionalization complexities.

Broadly, a nanocomposite that overcomes the deficiencies of the prior art is described herein. The nanocomposite comprises a Pani-carbon (Pani/C) nanocomposite with a metal, e.g., Ni²⁺, complexation system comprising, e.g., a tetradentate ligand such as nitrilotriacetic acid (NTA). In some embodiments, Ni²⁺ is a multifunctional and low-cost nanoparticle for nitrate binding, while NTA-functionalized polymers can be easily adsorbed on amorphous carbon. NTA is a tetradentate ligand that forms a hexagonal complex with a divalent metal ion (e.g., Ni²⁺) without affecting the electrochemical performance of Pani. The present inventors are unaware of any reporting of the use of Ni-NTA incorporated Pani/C nanocomposites for the detection of nitrate in water. Further, an assembly comprising a working electrode comprising a Ni-NTA incorporated Pani/C nanocomposite (also referred to as Ni@Pani/C herein) coating is described. In addition to this working electrode, a reference electrode and counter electrode can be used to complete a three-cell electrochemical system.

Accordingly, in a first aspect, a nanocomposite comprising, consisting of, or consisting essentially of nitrate-binding metal ion complexes incorporated in and/or on polyaniline/carbon nanostructure (Pani/C) is described. In some embodiments, the nitrate-binding metal complex comprises at least one metal ion and at least one tetradentate ligand that forms a hexagonal complex with the divalent metal ion. In some embodiments, the at least one metal ion is divalent. In some embodiments, the at least one divalent metal ion is selected from Zn(II), Co(II), Ca(II), Cu(II), Mg(II), Mn(II), Fe(II), Cd(II), Ba(II), Sr(II), Pb(II), Pt(II), Ti(II), Pd(II), and Ni(II). In some embodiments, the at least one metal ion is a divalent nickel ion. In some embodiments, the at least one tetradentate ligand is selected from ethylene diamine diacetic acid (EDDA), iminodiacetic acid, tris(carboxymethyl)ethylenediamine, ethylenediaminetetraacetic acid, diethylene triamine pentaacetic acid, DOTA (1,4,7,10-tetraazacyclododecane-1,4,7,10-tetraacetic acid), and nitrilotriacetic acid (NTA). In some embodiments, the tetradentate ligand is nitrilotriacetic acid (NTA). In some embodiments, the molar ratio of the at least one metal ion to the at least one tetradentate ligand is in a range from about 1:5 to about 5:1, or about 1:4 to about 4:1, or about 1:3 to about 3:1, or about 1:2 to about 2:1, or about 1:1.5 to about 1.5:1, or about 1:1.2 to about 1.2:1, or about 1:1.1 to about 1.1 to about 1, or about 1:1.

In some embodiments, the carbon is amorphous. In some embodiments, the carbon is carbon black. In some embodiments, the carbon black is amorphous. In some embodiments, the nanocomposite comprises interconnected granular nanoparticles forming porous channels. In some embodiments, the Pani is substantially uniformly distributed and non-aggregated on the carbon black. In some embodiments, the nanocomposite comprises a nitrate-binding Ni-NTA complex incorporated in and/or on a Pani/C nanostructure, wherein the nickel is Ni²⁺ (e.g., Ni@Pani/C). In some embodiments, the polyaniline/carbon nanostructure comprises about 20 wt % to about 70 wt % polyaniline, or about 30 wt % to about 70 wt % polyaniline, or about 40 wt % to about 70 wt % polyaniline, or about 50 wt % to about 70 wt % polyaniline, or about 60 wt % to about 70 wt % polyaniline, or about 63 wt % to about 67 wt % polyaniline, based on the total weight of the Pani/C nanostructure. In some embodiments, the polyaniline/carbon nanostructure comprises about 65 wt % polyaniline. In some embodiments, the weight percent of the Pani/C in the nanocomposite is about 60 wt % to about 99 wt %, or about 70 wt % to about 98 wt %, or about 80 wt % to about 95 wt %, based on the total weight of the nanocomposite.

In some embodiments, the nanocomposite comprises, consists of, or consists essentially of nitrate-binding metal ion complexes incorporated in and/or on polyaniline/carbon black nanostructure (Pani/C). In some embodiments, the nanocomposite further comprises at least one additional material, e.g., a binder, a filler, a conductive agent, inert materials, or any combination thereof.

In some embodiments, the nanocomposite, i.e., Ni@Pani/C, can be suspended in at least one solvent, e.g., polyvinyl acetate (PVA) in isopropyl alcohol, and then spray coated onto a graphite-containing sensor electrode and dried thereon to produce a working electrode. In some other embodiments, a graphite-containing sensor electrode comprising the nanocomposite, i.e., Ni@Pani/C, adhered thereto is described.

In a second aspect, a device is disclosed herein that is an ecofriendly microfluidic cassette comprising the nanocomposite of the first aspect, i.e., Ni@Pani/C, as a working electrode material that can be used for nitrate detection in water. The innovation has been demonstrated in two parts namely adaptation of conventional macro cell into a microfluidic cassette made of acrylic sheets (PMMA) followed by adaptation of the PMMA microfluidic cassette into a wood-based microfluidic cassette. The nanocomposite of the first aspect has shown high specificity towards nitrate and high stability over long shelf life, pH and temperature.

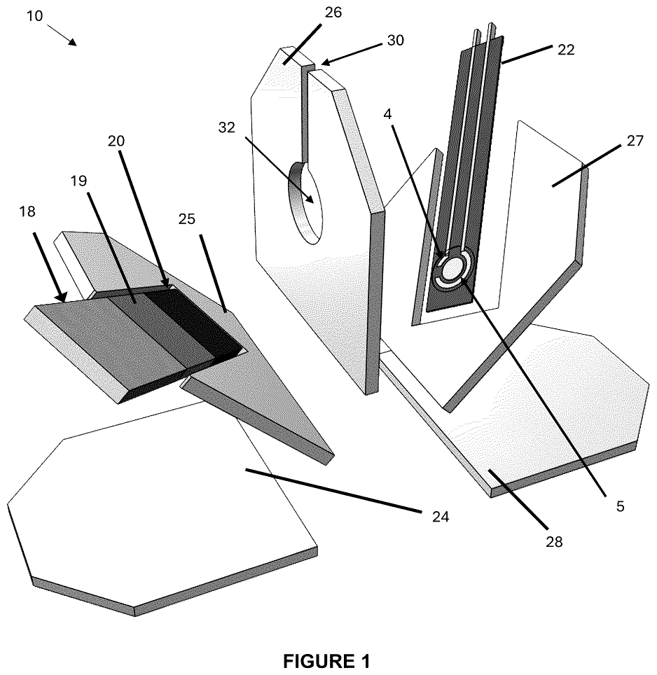

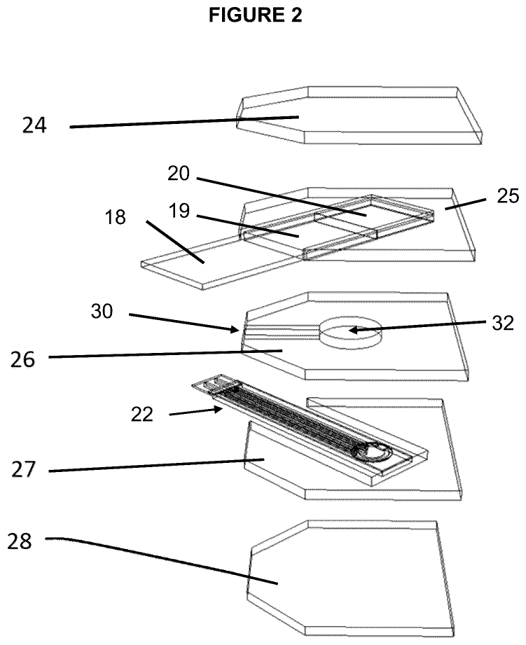

In some embodiments, a PMMA microfluidic cassette (10) is described, as illustrated in FIGS. 1 and 2. The PMMA microfluidic cassette was used to show proof of concept for first step adaptation from macro cell to a microfluidic cell. As shown in FIGS. 1 and 2, an electrode comprises graphite material (18) coated with the nanocomposite of the first aspect, i.e., Ni@Pani/C, as the working electrode (20) with an insulator coating (19), e.g., polystyrene in chloroform. The electrode (22) comprises a counter electrode (4) and a reference electrode (5), e.g., a commercially available screen-printed PET electrode, completes a three-electrode setup. In some embodiments, the counter electrode comprises platinum or carbon, e.g., screen printed conducting carbon, and the reference electrode comprises Ag/AgCl. In some embodiments, the PMMA microfluidic cassette comprises five layers: two end-layers (24, 28), middle connector layer (26) comprising a microwell (32) and an inlet/outlet (30) for the sample (i.e., sample possibly comprising nitrate), a layer (25) that has an engraved portion or a first bore to accommodate the working electrode, and a layer (27) that has an engraved portion or a second bore to accommodate electrode (22) comprising the counter and reference electrodes. It should be appreciated by the person skilled in the art that the size and shape of the microfluidic cassette, as well as layers (25) and (27) can be modified as needed, e.g., to accommodate the size and shape of the respective electrodes. It should be appreciated by the person skilled in the art that the electrode (22) does not have to be limited to a commercially available one but can be fabricated based on the requirements of the user or can be replaced with any type of counter and reference/pseudo reference electrodes. It is also to be appreciated that although the PMMA microfluidic cassette is shown as comprising five layers, layers (27) and (28) can be one monolithic layer (e.g., a second bore can be engraved in the layer to accommodate electrode (22)) or layers (24) and (25) can be one monolithic layer (e.g., a first bore can be engraved in the layer to accommodate the working electrode), or both, and as such a three- or four-layer PMMA microfluidic cassette is easily envisioned. Further, it should also be appreciated that the entire PMMA microfluidic device can be one monolithic piece, produced, e.g., using 3D printing, as readily understood by the person skilled in the art.

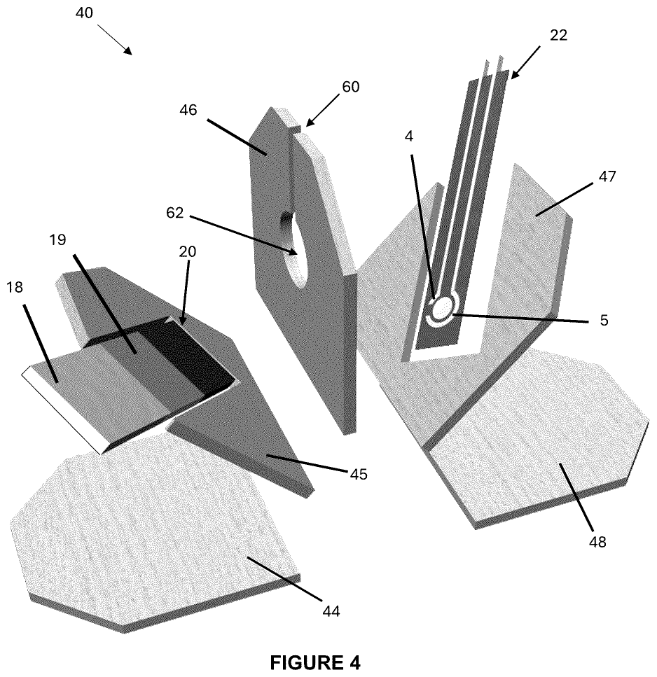

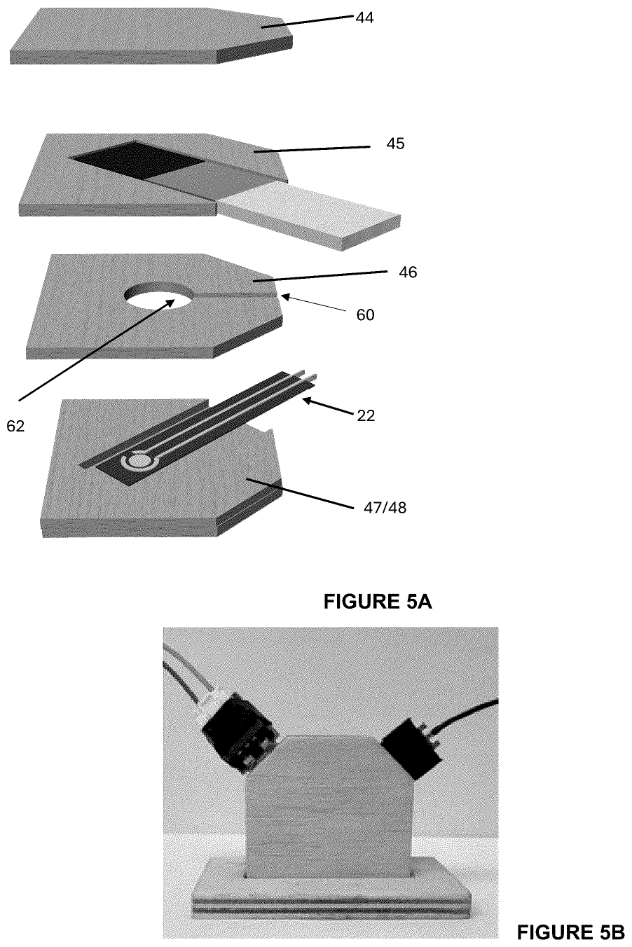

Based on the PMMA microfluidic cassette of FIGS. 1 and 2, an embodiment of a wood-based microfluidic cassette (WMC) (40) housing a three-electrode electrochemical cell is described. The arrangement of the WMC is analogous to the PMMA microfluidic cassette, as shown in FIGS. 4 and 5. An electrode comprises graphite material (18) coated with the nanocomposite of the first aspect, i.e., Ni@Pani/C, as the working electrode (20) with an insulator coating (19), e.g., polystyrene in chloroform. The electrode (22) comprising a counter electrode (4) and a reference electrode (5), e.g., a commercially available screen-printed PET electrode, completes the three-electrode setup. In some embodiments, the counter electrode comprises platinum or carbon, e.g., screen printed conducting carbon, and the reference electrode comprises Ag/AgCl. In some embodiments, as shown in FIG. 4, the WMC comprises four layers: a layer (45) with an engraved portion or first bore that accommodates the working electrode, middle connector layer (46) comprising a microwell (62) and an inlet/outlet (60) for the sample (i.e., sample possibly comprising nitrate), a layer (47) that has an engraved portion or second bore that accommodates the electrode (22), and an end layer (48). In some embodiments, as shown in FIG. 5A, the WMC comprises five layers: two end-layers (44, 48), middle connector layer (46) comprising a microwell (62) and an inlet/outlet (60) for the sample (i.e., sample possibly comprising nitrate), a layer (45) that has an engraved portion or a first bore to accommodate the working electrode, and a layer (47) that has an engraved portion or a second bore to accommodate electrode (22) comprising the counter and reference electrodes. It should be appreciated by the person skilled in the art that the size and shape of the WMC, as well as layers (45) and (47) can be modified as needed, e.g., to accommodate the size and shape of the respective electrodes. It should be appreciated by the person skilled in the art that the electrode (22) does not have to be limited to a commercially available one but can be fabricated based on the requirements of the user or can be replaced with any type of counter and reference/pseudo reference electrodes. It is also to be appreciated that although the WMC is shown in FIG. 4 as comprising five layers, layers (47) and (48) can be one monolithic layer (see for example FIG. 5A) or layers (44) and (45) can be one layer, or both, and as such a three- or four-layer WMC is easily envisioned. Further, it should also be appreciated that the entire WMC can be one monolithic piece, as readily understood by the person skilled in the art.

Regardless of the material of construction for the microfluidic cassette, in some embodiments, the inlet/outlet are the same channel for manual fill/discard (e.g., as shown). Regardless of the material of construction, in some embodiments, the inlet and outlet can be different channels for controlled fill/discard of reagents and analytes with or without valves (not shown), e.g., a flow-cell for automated continuous monitoring of nitrate. Regardless of the material of construction, in some embodiments, the engineered microfluidic cassette has a three-electrode cell assembled in the microwell in the same plane or in a different plane facing each other (e.g., as shown), so that the individual working and counter/reference electrodes are easily replaceable thereby facilitating sustainable low-cost management practices. Regardless of the material of construction, in some embodiments, the microwell is accessible from two directions for easier replaceability of the electrodes. Regardless of the material of construction, it should be appreciated that although the reference and counter electrodes are described as being on the same electrode (22), it is envisioned that a separate reference electrode and a separate counter electrode can be accommodated in the microfluidic cassette. Regardless of the material of construction, the microwell can have a volume in a range from about 100 μL to about 1000 μL, or about 200 μL to about 900 μL, or about 300 μL to about 800 μL, or about 400 μL to about 600 μL, or about 400 μL to about 500 μL. Regardless of the material of construction, the first bore that accommodates the working electrode ensures that the working electrode is in contact with the contents of the microwell and the second bore that accommodates the reference electrode and counter electrode ensures that the reference electrode and the counter electrode are in contact with the contents of the microwell and in proximity to the working electrode. Regardless of the material of construction, the first and second bores permit electrical connection of the working electrode and the reference and counter electrodes with an analytical device for sensing the presence of nitrate in the water sample. Regardless of the material of construction, liquid cannot escape from the microwell via the first and second bores comprising the respective electrodes.

Electrical connections for the electrodes of the electrochemical cell are well known in the art. Further, analytical techniques and electrochemical analyzers that can be used to convert the impedance or electrical current data of the electrochemical cell to nitrate concentrations are also well known in the art and will not be discussed further herein.

Accordingly, in some embodiments of the second aspect, an apparatus for detecting nitrate (NO₃⁻) ions in a water sample is described, said apparatus comprising:
a microfluidic cassette comprising:
   a microwell having an inlet and an outlet for introduction of the water sample to the microwell and removal of same therefrom;
   a first bore that accommodates a working electrode such that the working electrode is in contact with the contents of the microwell; and
   a second bore that accommodates a reference electrode and a counter electrode, wherein the reference electrode and the counter electrode are in contact with the contents of the microwell and in proximity to the working electrode.
      In some embodiments, the working electrode comprises a graphite-containing sensor electrode comprising a nanocomposite adhered thereto, wherein the nanocomposite comprises comprising nitrate-binding metal ion complexes incorporated in and/or on a polyaniline/carbon (Pani/C) nanostructure.

In a third aspect, a method of measuring a concentration of nitrate ions in a water sample is described, said method comprising:
introducing a liquid buffer to the inlet of an apparatus for detecting nitrate (NO₃⁻) ions in a water sample;
introducing the water sample to the apparatus by dipping the cassette in water or by introducing the water sample to the inlet of the cassette; and
measuring the impedance or electrical current using an electrochemical analyzer and converting same to nitrate concentration.
   In some embodiments, the apparatus comprises a microfluidic cassette comprising: a microwell having an inlet and an outlet for introduction of the water sample to the microwell and removal of same therefrom; a first bore that accommodates a working electrode such that the working electrode is in contact with the contents of the microwell; and a second bore that accommodates a reference electrode and a counter electrode, wherein the reference electrode and the counter electrode are in contact with the contents of the microwell and in proximity to the working electrode. In some embodiments, the working electrode comprises a graphite-containing sensor electrode comprising a nanocomposite adhered thereto, wherein the nanocomposite comprises comprising nitrate-binding metal ion complexes incorporated in and/or on a polyaniline/carbon (Pani/C) nanostructure.

### Example 1

### A. Materials

Potassium Ferrocyanide (K₄Fe(CN)₆, 99%), Potassium Chloride (KCl, 99%), Sodium Sulphate (Na₂SO₄, 99%), Sulphuric Acid (H₂SO₄, 99%), Phosphoric Acid (H₃PO₄, 99%), Polyaniline (emeraldine salt) composite (20 wt. % polyaniline on carbon black), aniline, ammonium persulphate, isopropyl alcohol (IPA), ethanol were procured from Sigma-Aldrich. Nitrilotriacetic acid was procured from thermos scientific and Nickel chloride from CAROLINA. Phosphate Buffer Saline (PBS, 99%) was obtained from Merck. A 1000 ppm Nitrate standard (as NO₃, 99%) was procured from RICCA and diluted to make nitrate solutions for analysis. The purchased chemicals were of analytical grade and were used without further processing. All the solutions for analysis were prepared in high performance liquid chromatography (HPLC) grade deionized water. Clarex thin poly(methyl methacrylate) (PMMA) sheets of 0.5 mm and 1 mm were from Astra products. Unfinished craft balsa wood sheets of 2 mm thickness were used for making wood microfluidic cassettes.

### B. Methods of Synthesis and Characterization of the Nanomaterials

A commercial composite of Polyaniline emeraldine salt with 20 wt % polyaniline on carbon black (designated as Pani/C(20)) was used for synthesizing 65 wt % polyaniline on carbon black (designated as Pani/C(65)) through an in-situ chemical polymerization method with varying weight ratios of aniline. In each synthesis set, purified aniline was introduced into a 100 mg suspension of Pani/C(20) and subjected to sonication for approximately 3 hours. The solution was then stirred in an ice bath at a temperature of 0-4° C. Subsequently, a pre-cooled solution of ammonium persulfate (APS) with a molar ratio of aniline to APS of 1:4 was added dropwise into the above solution under vigorous stirring and left to stir overnight while maintaining the temperature at 0-4° C. The polymerized product, denoted as Pani/C(65) was collected after centrifugation, washed with water and alcohol, and then dried under vacuum. Next, nickel chloride and nitrilotriacetic acid (NTA) were separately dissolved in alcohol and mixed at a molar ratio of 1:1 with a fixed content of Pani/C(65), wherein the fixed content of Pani/C(65) is 90 wt % relative to the total weight of the total nanocomposite (i.e., for every 90 mg of Pani/C(65), 10 mg of (Ni-NTA) was used) under constant stirring over 5 hours [Lori, 2006]. The resulting product, Ni@Pani/C, was collected through centrifugation, washed with alcohol, and dried under vacuum overnight.

Powder samples were analyzed by Rigaku X-ray Diffractometer. A Hitachi HT7800 120 kV microscope was used to conduct transmission electron microscopy (TEM). FEI Nova NanoSEM 450 field-emission scanning electron microscope also equipped with EDS and EBSD capability (Oxford Aztec) was used for morphology and elemental analysis (Energy Dispersive X-ray analysis). Powdered sample on conductive carbon tape was used for FESEM. Further, powder samples were analyzed by Raman Spectroscopy on Renishaw in Via Raman microscope equipped with a laser having wavelength 514 nm. Fourier Transform Infra-red (FTIR) spectra of powder samples were recorded on a Perkin-Elmer FTIR spectrum BX spectrometer.

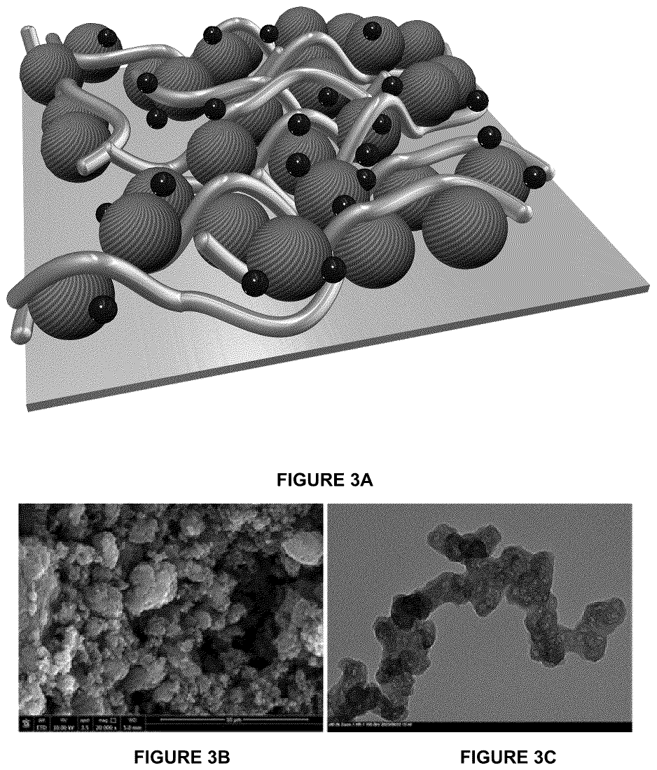

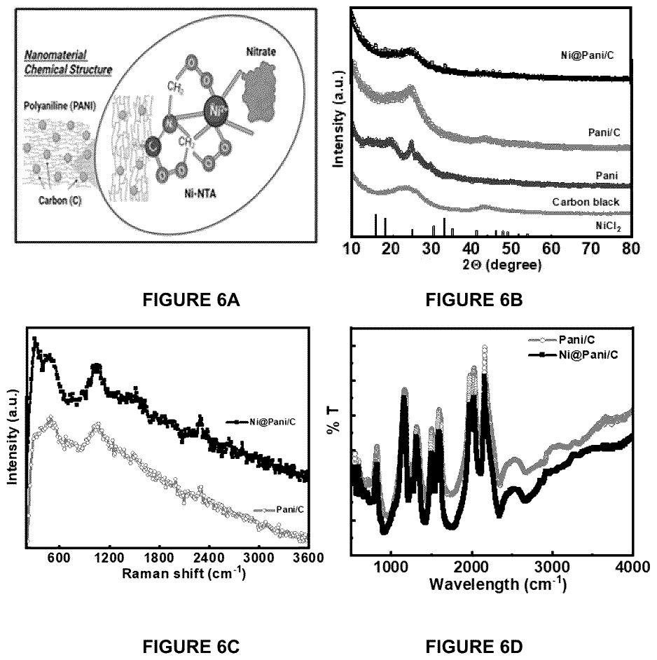

FIG. 3A illustrates a schematic representation of Ni@Pani/C coated on a graphite electrode (18). The nanocomposite comprises polyaniline (Pani) (16), carbon black (15) and Nickel ions (14). Surface morphology of Ni@Pani/C nanocomposite, as investigated by SEM, exhibits uniform sized pseudo-spherical interconnected granular nanoparticles forming porous channels (see, FIG. 3B). Furthermore, elemental mapping was done using EDX over a large sample size and the result shows uniform and continuous distribution of carbon (C), oxygen (O), nitrogen (N), with spatially distributed nickel (Ni) (not shown). The TEM image demonstrated inter-woven circular structure of the carbon with uniformly distributed (or non-aggregated) Pani nanoparticles on the carbon (Pani/C) (FIG. 3C). Polyaniline nanoparticles are dispersed on carbon without being aggregated and without blocking the porous channels in the entangled material. In addition, the XRD results confirmed the presence of carbon, Pani and nickel in its Ni²⁺ complex state (FIG. 6B). These are further ascertained by Raman (FIG. 6C) and FTIR spectroscopy (FIG. 6D). Based on the analysis results, a possible structure of the prepared nanocomposite (Ni@Pani/C) is illustrated in FIG. 6A.

For the fabrication of the sensing electrodes, 5 mg of sample was sonicated with 10 wt % polyvinyl acetate (PVA) in isopropyl alcohol for 30 minutes. The resulting suspension was deposited on a polished glassy carbon surface, followed by thermal treatment at 50° C. for 1 hour. This was used as a working electrode in the three-electrode electrochemical setup. For microfluidic sensor, the fabrication process for working electrodes followed a similar protocol. Initially, 5 mg of the sample was sonicated with 5 μL of PVA binder in isopropyl alcohol for 30 minutes. The resulting mixture was then spray-coated onto the surfaces of graphite plates, each measuring 1 cm×1 cm. Subsequently, the coated graphite plates were dried in an oven at 80° C. for 1 h to ensure proper adhesion of the active material. To determine the amount of active material loaded onto each graphite plate, the plates were weighed before and after the spraying/drying process. The weight of the active material was found to be in the range of 1.1-1.5 mg/cm².

### C. Fabrication of Microfluidic Cassette

The layers of cassettes were designed as 2D drawings in CorelDraw. These designs were printed on PMMA or wood sheets using a CO₂ laser engraver with >9.3 μm CO₂ laser with 10-75 watt power and 2.0 lens module [Andar, 2019; Al-Adhami, 2017]. Laser settings such as laser power and speed were altered to cut and engrave the sheets. For optimization studies, 0.5-2 mm PMMA sheets were used to form layers of the microfluidic cassette. For bonding PMMA cassettes, the layers were cleaned with 100% alcohol and lint free tissue. After cleaning, the layers were aligned together with 100% alcohol sprayed between layers and loaded onto custom-designed microwave safe vise to hold the layer together under pressure [Hasan, 2022]. This was placed in a domestic microwave for two minutes for heating and binding the layers into a cassette.

### D. Methodology of Electrochemical Nitrate Sensing

Electrochemical tests were performed in a conventional three-electrode cell with a CHI604D workstation. Screen printed conducting carbon, Ag/AgCl and Ni@Pani/C nanocomposite (with 10 wt. % binder polyvinyl acetate in acetone) coated (1 cm²) graphite plates were used as counter, reference and working electrode, respectively. Cyclic voltammetry studies of Ni@Pani/C nanocomposite graphite electrode were carried out reversibly between-0.8 V to 0.8 V. The set up was purged with N₂ gas for 30 minutes before starting the experiments to get rid of interference caused by dissolved oxygen. To understand the ionic and electronic conduction across electrolyte/electrode surface, electrochemical impedance studies (EIS) were performed at a voltage of −0.4 V and for a frequency range of 1 MHz-0.1 Hz. To understand the effect of diffusion, Warburg impedance coefficient was obtained by simulating a Nyquist plot in an EIS spectrum analyzer (EISSA). Charge transfer resistance (R_{CT}) and corresponding solution resistance (R_{S}) values were fitted to an equivalent Randles Cell circuit in EISSA where a double layer Capacitance was in parallel, and together they were in series with R_{S} and Warburg impedance. Powell algorithm, 300 iterations, n1≈1 and P1=1e⁻⁵ to 1e⁻⁷ parameters were chosen to get a good fit.

Electrochemical studies were performed in 100 mM K₄Fe(CN)₆, 0.1 M KCl and 50 mM PBS electrolyte. The sensing behavior was monitored at different pH values, by adjusting the pH of the electrolyte to 4.5, 6 and 7 using 0.1% (V/V) Phosphoric Acid. The microfluidic set up utilized only 400-500 μL of electrolyte and nitrate sensing was performed by successive addition of aliquots for a concentration range of 0.8 mg/L to 100 mg/L. Selectivity of the electrode towards different ions was assessed by performing the EIS of 4 mg/L of commonly occurring ions in water such as acetate, bicarbonate, sulphate, and ammonium with respect to nitrate. Furthermore, stability of sensing at different temperatures was tested by monitoring the EIS on the addition of 2 mg/L nitrate by maintaining the electrolyte temperature from 5° C. to 65° C. Further, the stability of the coated materials was tested by measuring nitrate sensing over a period of 30 days.

To check the real-time applicability of the sensors, stream samples containing nitrate were collected from the streams containing nitrate in the Baltimore, Maryland, USA, region. Stream sampling was carried out by walking upstream from Dead Run (DR 7) watershed within the Gunpowder-Patapsco drainage to the Chesapeake Bay, Gwynns Falls and Alexander Avenue over a period of approximately 5 hr. 60-ml HDPE Nalgene bottles were rinsed 3 times with stream water then submerged in the stream for sampling and capped underwater, allowing no head space, and kept on ice during the sampling period. Samples were used without filtering as well as subsequently filtered in the laboratory within 24 hr using a 0.45-micron glass microfiber filter, always keeping samples on ice. Standard commercial optical absorbance-based device: Submersible UV Nitrate Analyser (SUNA V2) from Sea-Bird Scientific is utilized for nitrate detection for comparison with the nitrate sensor described herein. Nitrate sensing ability of the electrode in the presence of the interfering ions in these water samples was tested.

### E. Optimization of Experimental Methods

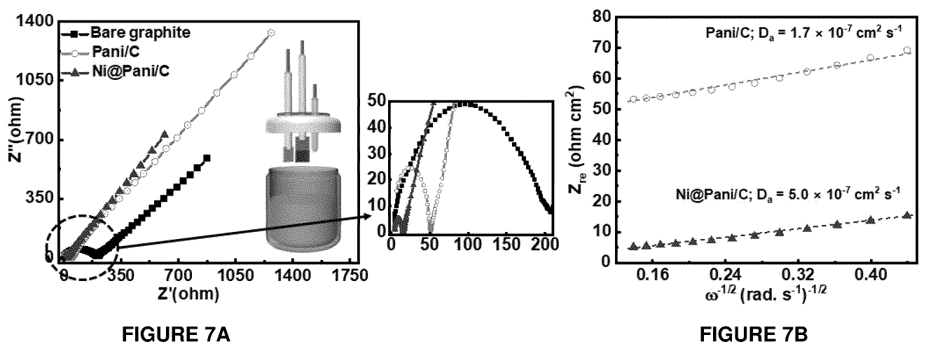

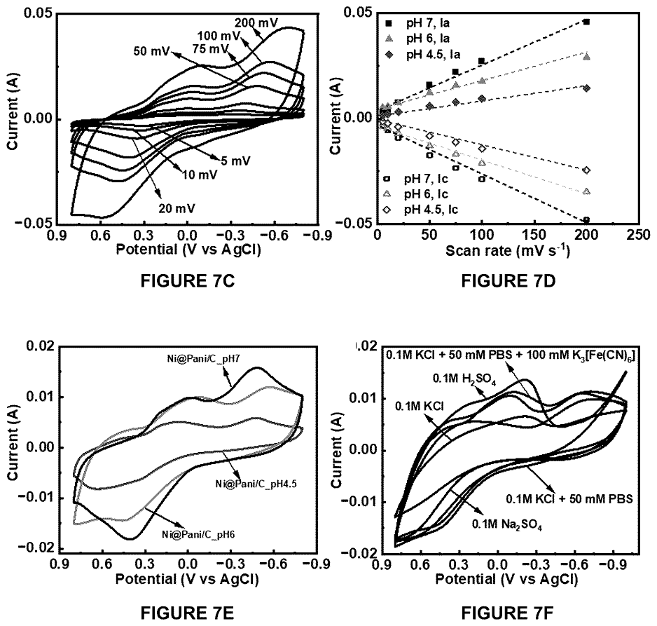

Optimization of experimental conditions was performed in a three-electrode cell setup, for example as illustrated in the 3D schematic shown in FIG. 7A inset. This characterization was carried out in a N₂-saturated 0.1 M phosphate buffer solution (PBS) with added mediator [Fe(CN)₆] (5 mM) and KCl (0.1 M) at a pH of 7.0. As depicted in FIG. 7A, impedance results from Nyquist plots observed in the magnified image in FIG. 2A outset confirms the incorporation of Ni²⁺ in Pani/C which is evident from a smaller semi-circle in the high frequency region, as well as a lower R_{CT} value (R_{CT}≈8Ω) compared to Pani/C (R_{CT}≈44Ω) and bare graphite electrode (R_{CT}≈210Ω). These findings suggest that Ni@Pani/C may enhance the transport of the ferricyanide/ferrocyanide redox couple [Ujjain, 2015]. This phenomenon is further corroborated by the apparent diffusion coefficient (Dₐ) of ions at the Ni@Pani/C/electrolyte interface that is measured to be 5.0×10⁻⁷ cm² s⁻¹, which is three times higher than the value of 1.7×10⁻⁷ cm² s⁻¹ observed for the Pani/C electrode (FIG. 7B). Consequently, this indicates an enhanced ion diffusibility both vertically and horizontally within the Ni@Pani/C film. The combination of low charge transfer resistance (R_{CT}), high conductivity, and increased diffusion coefficient in the Ni@Pani/C film facilitates accelerated electron transfer between the electrochemical probe [Fe(CN)₆]^{3-/4-} and the electrode.

The impact of scan rate on the electrochemical behavior of the Ni@Pani/C electrode was investigated, as shown in FIGS. 7C-7D. It was observed that both the anodic peak (Eₚₐ) and cathodic peak (E_{pc}) undergo slight shifts in the positive and negative directions, respectively. Additionally, the peak currents, Iₚₐ, and I_{pc} show linear proportionality to the scan rate at various pHs of the same electrolyte, indicative of a surface-controlled reversible process. Moreover, the redox peak position and currents exhibit variations with changes in the pH of the electrolyte (FIG. 7E). Among the tested conditions, the best performance was achieved in a 50 mM PBS, 5 mM [Fe(CN)₆]^{3-/4-}, and 0.1M KCl at 50 mV/s, as illustrated in FIG. 7F.

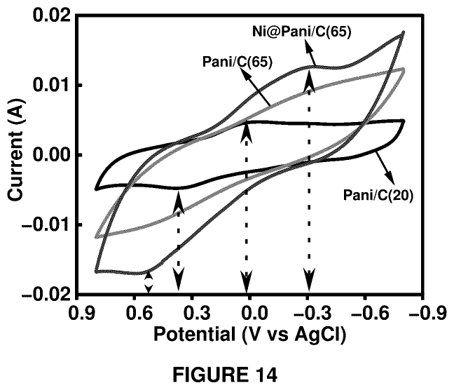

In terms of nitrate sensing activity, Pani/C(20) less than Pani/C(65) less than Ni@Pani/C(65) (see, FIG. 14). With the increasing content of Pani and the further introduction of Ni, the redox potential of the composite shifts closer to that of nitrate, resulting in improved nitrate sensing.

### F. Nitrate Sensing with the Nanomaterial in Conventional 3-Electrode Electrochemical Cell Macro System

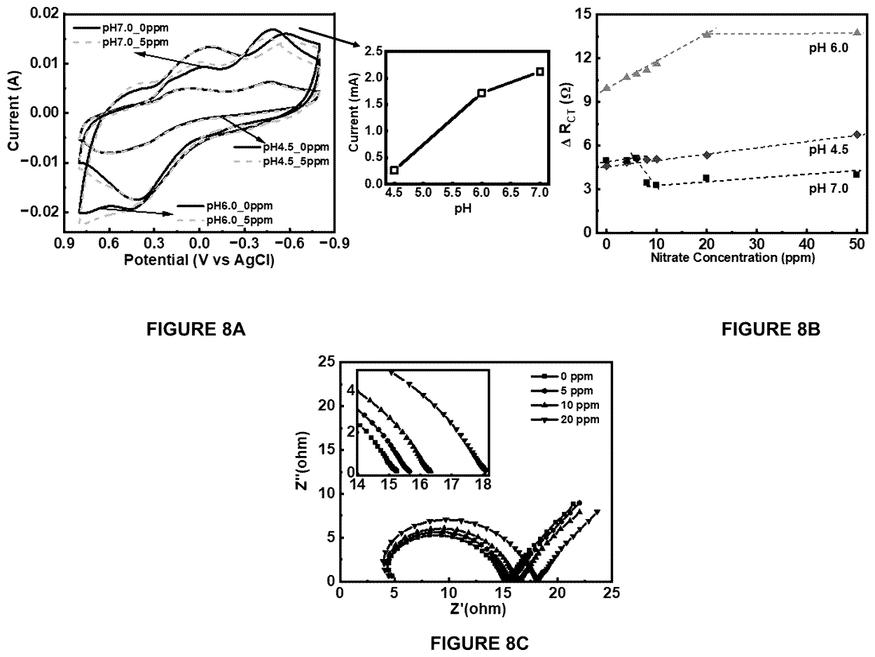

The electrocatalytic activity of the Ni@Pani/C nanocomposite was further investigated for NO₃⁻ sensing using a three-electrode cell assembly, e.g., as depicted in the FIG. 7A insert. FIG. 8A illustrates the cyclic voltammetry (CV) of the Ni@Pani/C sensor before and after the injection of an aliquot of NO₃⁻ (5 ppm) at 50 mV s⁻¹, conducted at pH 4.5, 6.0, and 7.0. Upon the addition of NO₃⁻, a reduction in oxidation peak currents around-0.45 V is observed. This decrease in current is comparable for both pH 6.0 and pH 7.0. However, at pH 6.0, the decrement in peak current remains continuous with successive additions of NO₃⁻. Consequently, EIS sensing is performed for the Ni@Pani/C sensor at −0.45 V upon successive injection of different concentrations (1-50 ppm) of NO₃⁻ at the various pHs (FIG. 8C). It is evident that the observed change in charge transfer resistance (R_{ct}) with increasing concentrations of NO₃⁻ is most prominent for the sensor operating at pH 6.0 (FIG. 8B). Based on all of the above considerations, a buffer composition of 0.1 M KCl+50 mM PBS+100 mM K₃Fe(CN)₆ electrolyte with pH 6.0 was chosen for nitrate sensing in the Center for Advanced Sensor Technology (CAST) microfluidic (CAST-MF) electrochemical cassette.

### G. Nitrate Sensing with the Nanomaterial in a Three-Electrode Cell PMMA Microfluidic System

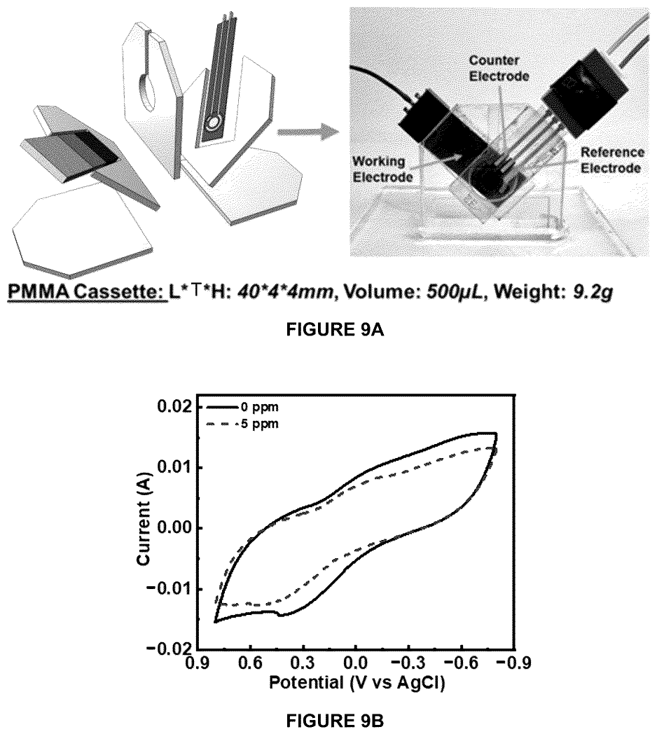

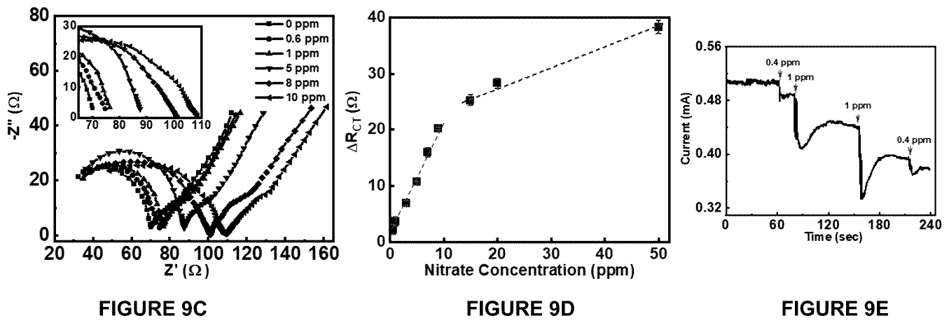

Based on the aforementioned investigations, similar sensing experiments are performed on the PMMA based microfluidic Ni@Pani/C nanocomposite sensors (FIGS. 4, 5, and 9A). Miniaturization of the conventional macro-electrochemical setup reduced the operational volume from 75 mL to 0.5 mL, resulting in a significant decrease in the amount of reagents needed and minimizing the environmental footprint. The microfluidic setup provided a faster response time due to enhanced ionic interaction from closer electrode proximity. In addition to the fabricated working electrode (1 cm²), a pseudo-reference electrode and counter electrode from CAULYS were utilized to complete the three-electrode microfluidic electrochemical setup, with its sensing electrode unused as observed in FIG. 9B. Electrochemical analysis was conducted using 0.5 mL of electrolyte injected through the top slit (sample channel).

Although the change in current before and after the injection of an aliquot of NO₃⁻ (5 ppm) at 50 mV s⁻¹ is comparable to that of the conventional 3-electrode assembly, the continuous R_{CT} change with successive additions of NO₃⁻ was observed to be very prominent (FIG. 9C). The calibration curve (FIG. 9D) with increasing molar concentration of NO₃⁻ (0 ppm (mg/L) to 50 ppm) for Ni@Pani/C PMMA microfluidic sensor exhibits two slopes. In the lower concentration region (0.6 ppm to 10 ppm), the change in R_{CT}≈2.3±0.089 Ωcm⁻² ppm⁻¹, while higher concentration (15 ppm to 50 ppm), the change in R_{CT}≈0.36±0.043 Ωcm⁻² ppm⁻¹, with low detection limit (LDL) of 0.015 ppm. Both linear slopes could be used for sensing nitrate concentration based on ΔR_{CT} in unknown water samples. FIG. 9E shows an amperometric study using the Ni@Pani/C sensor with repeated 0.4 ppm and 1 ppm nitrate concentrations. The response time of the Ni@Pani/C sensor upon changing nitrate concentration was observed to be 20 seconds, which is quite rapid for a first-generation prototype and is comparable with the previously reported nitrate and nitrite based electrochemical sensors.

### H. Stability, Reproducibility and Reusability

In practice, the PMMA microfluidic sensor is loaded by introducing a liquid buffer/electrolyte to the inlet (30) using a dropper/pipette/automated inlet, and thereafter the water sample is loaded into the cassette by dipping the cassette in water or dropping the water sample into the cassette through the designated inlet or by a controlled flow through an automated inlet and outlet with/without valves. The change in impedance or current is measured using an electrochemical analyzer that is attached to the electrodes (see, for example, FIG. 9A for electrode attachments).

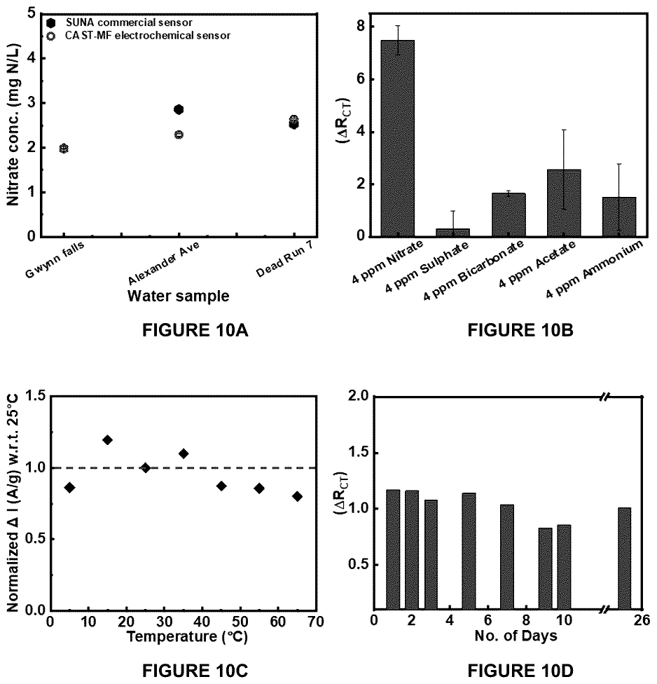

To assess the real-time applicability of the developed sensors, stream samples containing nitrate were collected from various streams in the Baltimore region, Maryland. The sampling locations included Dead Run (DR 7) watershed within the Gunpowder-Patapsco drainage, the Chesapeake Bay, Gwynns Falls, and Alexander Avenue. For comparison, a commercial optical absorbance-based device, the Submersible UV Nitrate Analyser (SUNA V2) from Sea-Bird Scientific, was also employed to determine nitrate levels in the collected stream samples. Samples were stored at −4° C. that were later thawed and analyzed within the first 24-48 hours. Results obtained from the Ni@Pani/C electrochemical sensor described herein showed good agreement with those obtained from the commercial SUNA device with an absolute error±0.6 mg N/L, thereby confirming the accuracy of the Ni@Pani/C sensor for nitrate detection in field samples and potential for commercialization, as depicted in FIG. 10A. To examine potential interference effects, various substances like sulphate, bicarbonate, acetate, and ammonium were investigated on the NO₃⁻ sensor based on microfluidic Ni@Pani/C, as shown in FIG. 10B. The addition of 4 ppm of these common interferences caused minimal change concerning the detection of NO₃⁻. Furthermore, the effect of temperature on the sensing performance was evaluated by measuring the relative activity of the sensor at different temperatures while maintaining fixed NO₃⁻ concentrations (FIG. 10C). The results demonstrate comparable sensor activity across the temperature range from 5 to 65° C. To determine the long-term stability of the sensor, EIS responses were recorded over 30 days with fixed concentrations of NO₃⁻ (FIG. 10D). The electrodes were stored in ambient air conditions when not in use. Remarkably, the sensor retained more than 95% of its initial response to NO₃⁻ even after 26 days of storage, indicating excellent long-term stability.

### I. Conclusions

A microfluidic electrochemical nitrate sensor based on a Ni@Pani/C nanocomposite is described, wherein the nitrate sensor is cost-effective, environmentally-friendly, and rapidly detects nitrate. Under optimized conditions, our sensor demonstrated a high sensitivity of 2.31±0.09 Ω/ppm/cm² across a wide concentration range of nitrate. It also showed a desirable low detection limit of 0.015 ppm and a swift response time under 20 seconds. It maintained repeatability over a wide temperature range (5°-65° C.) and exhibited consistent performance over an extended period. Moreover, the sensor displayed high specificity towards nitrate when tested against potential interferences (SO₄²⁻, C₂H₃O₂⁻, HCO₃⁻, NH₄⁺) and showed good reproducibility for the test water samples collected from various streams in Maryland, U.S.A. The microfluidic cassette design, scalable electrode fabrication, and the presence of Pani enables high selectivity, collectively contributing to the development of a highly sensitive nitrate sensor. A proportional increase in R_{CT} was observed, establishing a clear relationship between the electrochemical signals and increasing nitrate concentration even at sub-ppm levels. The sensor showed comparable results with the SUNA V2 commercial device for determining nitrate concentrations in stream samples from the Baltimore region, Maryland, U.S.A. The sensor could also operate with negligible interference from other ions in the field water samples. It demonstrated remarkable stability at room temperature and showed great repeatability in nitrate sensing over several days. Having a portable setup, rapid one-step analysis method, and ease of operation by non-trained personnel, the Ni@Pani/C sensor exhibits high potential for commercial application as an on-site nitrate analyzer. Overall, the sensor is competent in terms of cost (affordability per sample), dimensions (light, compact and hand-held), performance range (low detection limit), reliability (reproducible with least possible standard deviation), high precision and a rapid response rate.

### Example 2

### A. Proof of Concept-Nitrate Sensing with the Nanomaterial in Three-Electrode Cell Wood Microfluidic System

Plastic's durability in microfluidic electrochemical devices while advantageous, poses significant environmental challenges. As demand for sustainable and eco-friendly materials grows, wood-based microfluidic cassettes (WMC) offer superior biodegradability and a reduced environmental footprint.

Analogous sensing experiments were conducted on a wood-based microfluidic Ni@Pani/C nanocomposite sensors. The engineered WMC inlet-outlet assembly comprises a wood cassette which houses the three-electrode cell (e.g., as described in Example 1 with reference to the PMMA microfluidic cassette), a liquid electrolyte, and a Ni@Pani/C nanocomposite-coated working electrode. The areas of the wood cassette layers that would be exposed to reaction mixture (e.g., the microwell and inlet/outlet) can be coated with a solution of a hydrophobic material (e.g., polystyrene) dissolved in a solvent (e.g., chloroform) to create hydrophobic/waterproof walls. The overall dimensions of the WMC was L(ength)×Thickness)×H(eight) of 40×4×40 mm. The microwell has a volume of about 400-500 μL. In some embodiments, the WMC is made of ≤2 mm sheets of balsa or plywood and bonded together using polymer glue. The electrodes comprised a Ni@Pani/C nanocomposite coated (1 cm²) graphite plate for the working electrode and screen-printed conducting carbon and Ag/AgCl for the counter and reference electrode, respectively. In some embodiments, the engineered WMC has a three-electrode cell assembled in the microwell in the same plane or in a different plane facing each other (e.g., as shown), so that the individual working and counter/reference electrodes are easily replaceable thereby facilitating sustainable low-cost management practices. In some embodiments, the microwell is accessible from two directions for easier replaceability of the electrodes. In some embodiments, the inlet/outlet are the same channel for manual fill/discard (e.g., as shown). It should be appreciated that the inlet and outlet can be different channels for controlled fill/discard of reagents and analytes with or without valves (not shown), e.g., a flow-cell for automated continuous monitoring of nitrate. The thickness of each layer in the WMC can be optimized to accommodate all three electrodes in the preferred configurations, for example, in a range of about 0.5 mm to about 3 mm per layer.

In practice, the WMC is loaded by introducing a liquid buffer/electrolyte to the inlet (60) using a dropper/pipette/automated inlet, and thereafter the water sample is loaded into the cassette by dipping the cassette in water or dropping the water sample into the cassette through the designated inlet or by a controlled flow through an automated inlet and outlet with/without valves. The change in impedance or current is measured using an electrochemical analyzer that is attached to the electrodes (see, for example, FIG. 5B for electrode attachments).

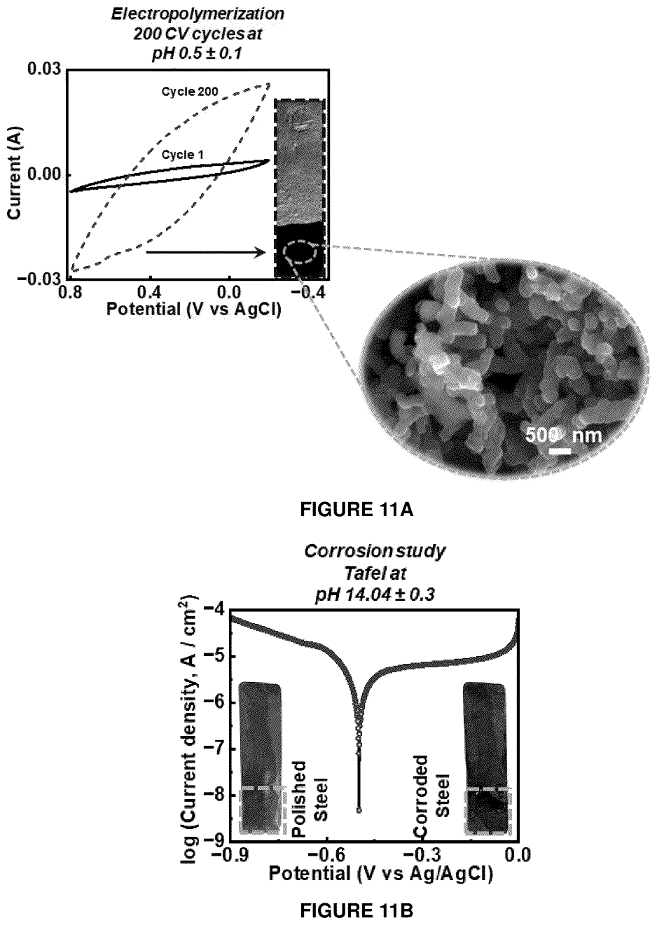

The WMC was adapted for electropolymerization of conducting polymer in an acidic medium (pH 0.5±0.1) with cyclic voltammetry (CV) (FIG. 11A) and corrosion current measurements by Tafel plot in a basic medium (pH 14.04±0.3) (FIG. 11B). In other words, the WMC demonstrates stability in performance over wide range of pH conditions.

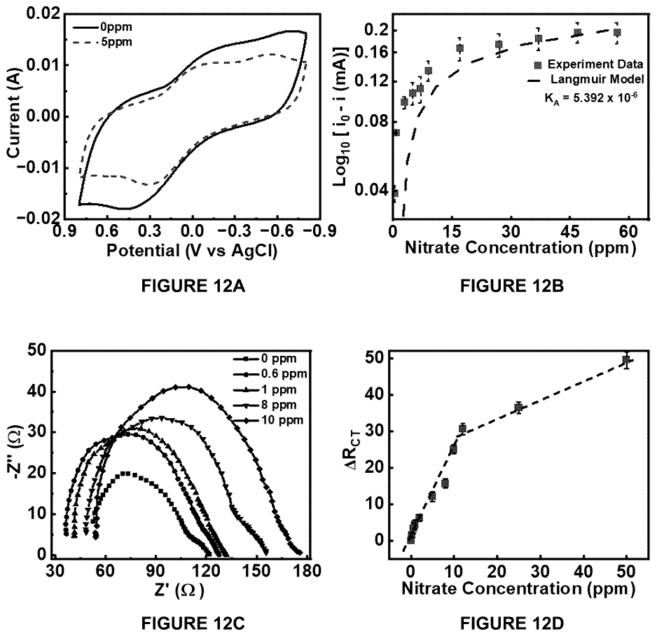

The electrochemical sensing of nitrate-containing solutions using the WMC was performed, including cyclic voltammetry at 50 mV s⁻¹ in absence and presence of 5 ppm NO₃⁻ (FIG. 12A), a Langmuir plot showing the adsorption of nitrate to the Ni@Pani/C nanocomposite over a range of 0.6-60 ppm nitrate (FIG. 12B), EIS Nyquist plots of Ni@Pani/C WMC upon successful injection of different concentrations of nitrate (0-10 ppm) in N₂ saturated 0.1 M PBS and pH 6.0 electrolyte at −0.45 V (FIG. 12C), and a calibration curve with increasing molar concentration of nitrate from 0.6-60 ppm (FIG. 12D) demonstrating high sensitivity in the concentration range. It can be seen that the WMC sensor demonstrates comparable and equally impressive performance to that of the PMMA microfluidic sensor.

### B. Stability, Reproducibility and Reusability

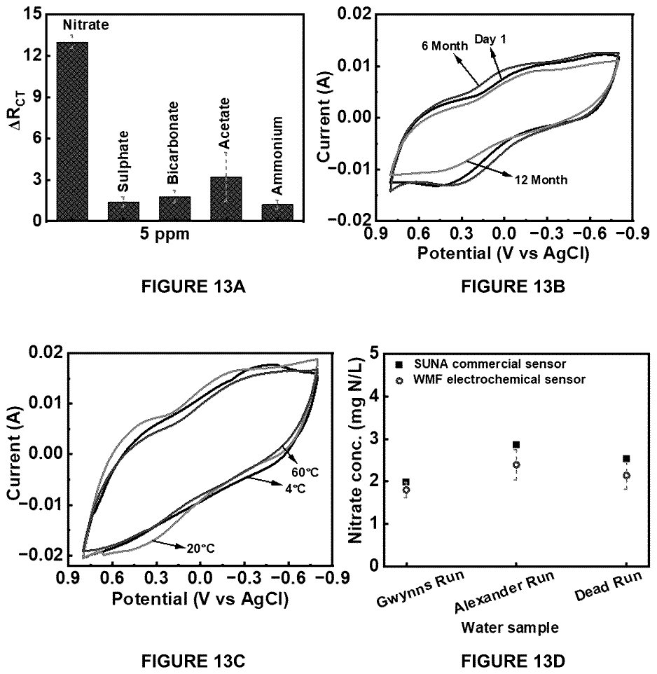

To assess the real-time applicability of the developed WMC sensors, stream samples containing nitrate were collected from various streams in the Baltimore region, Maryland. The sampling locations included Dead Run (DR 7) watershed within the Gunpowder-Patapsco drainage, the Chesapeake Bay, Gwynns Falls, and Alexander Avenue. For comparison, a commercial optical absorbance-based device, the Submersible UV Nitrate Analyser (SUNA V2) from Sea-Bird Scientific, was also employed to determine nitrate levels in the collected stream samples. The results obtained from the present sensor demonstrate equivalency in nitrate detection when compared to the commercial SUNA V2 sensor, as depicted in FIG. 13D. To examine potential interference effects, various substances like sulphate, bicarbonate, acetate, and ammonium were investigated on the WMC NO₃⁻ sensor based on microfluidic Ni@Pani/C, as shown in FIG. 13A. The addition of 4 ppm of these common interferences caused minimal change concerning the detection of NO₃. Furthermore, the effect of temperature on the sensing performance was evaluated by measuring the relative activity of the sensor at different temperatures while maintaining fixed 2 ppm NO₃⁻ concentrations (FIG. 13C). The results demonstrate comparable sensor activity across the temperature range from 4 to 60° C. FIG. 13B shows the long-term WMC stability performance for 12 months and various sampling over 100 cycles. In some embodiments, the WMC shows high structural stability for at least about 50 cycles. The electrodes were stored in ambient air conditions when not in use. Remarkably, the sensor retained more than 95% of its initial response to NO₃⁻ even after 12 months of storage, indicating excellent long-term stability.

### C. Conclusions

This example demonstrated the utility of wood microfluidic devices as a cost-effective and environmentally friendly approach for rapid nitrate detection and was intended to serve as a catalyst for diverse applications of wood-based devices, thereby reducing the reliance on plastics in similar applications. Importantly, the WMC sensor exhibits negligible interference from potential ions such as sulphate, bicarbonate, acetate, and ammonium. Designed for multiple sensing cycles, this sensor can be stored at room temperature in ambient air. Real-time applicability of the sensors for analyzing nitrate levels in stream samples collected from various locations in the Baltimore region, Maryland, USA, yields result that are comparable to those obtained using conventional device Submersible UV Nitrate Analyser (SUNA V2) from Sea-Bird Scientific.

Although the invention has been variously disclosed herein with reference to illustrative embodiments and features, it will be appreciated that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

### REFERENCES

Abascal, E; Gómez-Coma, L.; Ortiz, I.; Ortiz, A. Global diagnosis of nitrate pollution in ground water and review of removal technologies. Sci. Total. Environ., 2021, 810, 152233.
Al-Adhami, M.; Andar, A.; Tan, E.; Rao, G.; Kostov, Y. A solvent-based method to fabricate PMMA microfluidic devices. Chips and Tips, R S C 2017.
Andar, A.; Hasan, M-S.; Srinivasan, V.; Al-Adhami, M.; Gutierrez, E.; Bergenson, D.; Ge, X.; Tolosa, L.; Kostov, Y.; Rao, G. Wood Microfluidics. Analytical Chemistry, 2019, 91, 11004-11012.
Badea, M.; Amine, A.; Palleschi, G.; Moscone, D.; Volpe, G.; Curulli, A. New electrochemical sensors for detection of nitrites and nitrates. J. Electroanal. Chem. 2001, 509 (1), 66-72.
Bagheri, H.; Hajian, A.; Rezaei, M.; Shirzadmehr, A. Composite of Cu metal nanoparticles-multiwall carbon nanotubes-reduced graphene oxide as a novel and high performance platform of the electrochemical sensor for simultaneous determination of nitrite and nitrate. J. Hazard. Mater. 2017, 324, 762-772.
Banerjee, P.; Garai, P.; Saha, N. C.; Saha, S.; Sharma, P.; Maiti, A. K. A critical review on the effect of nitrate pollution in aquatic invertebrates and fish. Water, Air, & Soil Pollution 2023, 234, 333.
Butt, S. Simultaneous determination of nitrite and nitrate by normal phase ion-pair liquid chromatography. Talanta 2001, 55 (4), 789-797.
Charbaji, A.; Heidari-Bafroui, H.; Anagnostopoulos, C.; Faghri, M. A New Paper-Based Microfluidic Device for Improved Detection of Nitrate in Water. Sensors 2021, 21, 102.
Chen, X.; Pu, H.; Fu, Z.; Sui, X.; Chang, J.; Chen, J.; Mao, S. Real-time and selective detection of nitrates in water using graphene-based field-effect transistor sensors. Environ. Sci. Nano 2018, 5, 1990-1999.
Cogan, D.; Fay, C.; Boyle, D.; Osborne, C.; Kent, N.; Cleary, J.; Diamond, D. Development of a low cost microfluidic sensor for the direct determination of nitrate using chromotropic acid in natural waters. Analytical Methods, 2015, 13, 7, 5396.
Essousi, H.; Barhoumi, H.; Bibani, M.; Ktari, N.; Wendler, F.; Al-Hamry, A.; Kanoun, O. Ion-Imprinted Electrochemical Sensor Based on Copper Nanoparticles-Polyaniline Matrix for Nitrate Detection. J. Sensors 2019, 2019, 1-14.
Fossen Johnson, S. Methemoglobinemia: Infants at risk. Curr. Probl. Pediatr. Adolesc. Health Care 2019, 49 (3), 57-67.
Gal, C.; Frenzel, W.; Möller, J. Re-examination of the cadmium reduction method and optimization of conditions for the determination of nitrate by flow injection analysis. Microchimica Acta, 2004, 146, 155.
Glazier, S. A.; Campbell, E. R.; Campbell, W. H. Construction and Characterization of Nitrate Reductase-Based Amperometric Electrode and Nitrate Assay of Fertilizers and Drinking Water. Anal. Chem. 1998, 70 (8), 1511-1515.
Hasan, Md S.; Borhani, S., Ramamurthy, S. S.; Andar, A.; Ge, X.; Choa, F-S; Kostov, Y.; Rao, G. Microwave induced thermally assisted solvent-based bonding of biodegradable thermoplastics: an eco-friendly rapid approach for fabrication of microfluidic devices and analyte detection. Scientific Reports, 2022, 12, 16075.
Johnson, K. S.; Coletti, L. J. In situ ultraviolet spectrophotometry for high resolution and long-term monitoring of nitrate, bromide and bisulfide in the ocean. Deep Sea Res. Part I: Oceanogr. Res. Pap. 2002, 49 (7), 1291-1305.
Khanfar, M. F.; Al-Faqheri, W.; Al-Halhouli, A. Low cost lab on chip for colorimetric detection of nitrate in mineral water products. Sensors, 2017, 17, 10, 2345.
Kosa, S. A. M.; Khan, A. N.; Ahmed, S.; Aslam, M.; Bawazir, W. A.; Hameed, A.; Soomro, M. T. Strategic Electrochemical Determination of Nitrate over Polyaniline/Multi-Walled Carbon Nanotubes-Gum Arabic Architecture. Nanomaterials 2022, 12, 3542.
Le Goff, T.; Braven, J.; Ebdon, L.; Chilcott, N. P.; Scholefield, D.; Wood, J. W. An Accurate and Stable Nitrate-Selective Electrode for the In Situ Determination of Nitrate in Agricultural Drainage Waters. Analyst 2002, 127, 507-511.
Li, Z.; Liu, H.; Wang, D.; Zhang, M.; Yang, Y.; Ren, T-L. Recent advances in microfluidic sensors for nutrients detection in water. TrAC Trends in Analytical Chemistry, 2023, 158, 116790.
Lori, J. A.; Morrin, A.; Killard, A. J.; Smyth, M. R. Development and Characterization of Nickel-NTA-Polyaniline Modified Electrodes. Electroanalysis 2006, 18, 1, 77-81.
Mirvish, S. S. Role of N-Nitroso Compounds (NOC) and N-Nitrosation in Etiology of Gastric, Esophageal, Nasopharyngeal and Bladder Cancer and Contribution to Cancer of Known Exposures to NOC. Cancer Lett. 1995, 93, 17-48.
Obaideen, K.; Shehata, N.; Sayed, E. T.; Abdelkareem, M. A.; Mahmoud, M. S.; Olabi, A. G. The role of wastewater treatment in achieving sustainable development goals (SDGs) and sustainability guideline. Energy Nexus 2022, 7, 100112.
Pennino, M. J.; Compton, J. E.; Leibowitz, S. G. Trends in drinking water nitrate violations across the United States. Environ. Sci. Technol., 2017, 51, 22, 13450.
Snyder, L.; Potter, J. D.; McDowell, W. H. An evaluation of nitrate, fDOM, and turbidity sensors in New Hampshire streams. Water Resour. Res. 2018, 54, 2466-2479.
Ujjain, S. K.; Ahuja, P.; Sharma, R. K. Facile Preparation of Graphene Nanoribbon/Cobalt Coordination Polymer Nanohybrid for Non-enzymatic H₂O₂ Sensing by Dual Transduction: Electrochemical and Fluorescence. J. Mater. Chem. B 2015, 3, 7614-7622.
Wang, N.; Wang, R. Q.; Zhu, Y. A Novel Ion Chromatography Cycling-Column-Switching System for the Determination of Low-Level Chlorate and Nitrite in High Salt Matrices. J. Hazard. Mater. 2012, 235-236, 123-127.

What is claimed is:

1. A nanocomposite comprising nitrate-binding metal ion complexes incorporated in and/or on a polyaniline/carbon (Pani/C) nanostructure.

2. The nanocomposite of claim 1, wherein the metal ion is selected from at least one of Zn(II), Co(II), Ca(II), Cu(II), Mg(II), Mn(II), Fe(II), Cd(II), Ba(II), Sr(II), Pb(II), Pt(II), Ti(II), Pd(II), and Ni(II).

3. The nanocomposite of claim 1, wherein the metal ion complexes comprise at least one tetradentate ligand.

4. The nanocomposite of claim 3, wherein the at least one tetradentate ligand is selected from ethylene diamine diacetic acid (EDDA), iminodiacetic acid, tris(carboxymethyl)ethylenediamine, ethylenediaminetetraacetic acid, diethylene triamine pentaacetic acid, DOTA (1,4,7,10-tetraazacyclododecane-1,4,7,10-tetraacetic acid), and nitrilotriacetic acid (NTA).

5. The nanocomposite of claim 1, wherein the nanostructure comprises about 20 wt % to about 70 wt % polyaniline, based on the weight of the nanostructure.

6. The nanocomposite of claim 1, wherein the carbon is carbon black.

7. The nanocomposite of claim 1, wherein the weight percent of the Pani/C in the nanocomposite is about 60 wt % to about 99 wt %, based on the total weight of the nanocomposite.

8. A graphite-containing sensor electrode comprising the nanocomposite of claim 1 adhered thereto.

9. An apparatus for detecting nitrate (NO₃⁻) ions in a water sample, said apparatus comprising:
a microfluidic cassette comprising:
a microwell having an inlet and an outlet for introduction of the water sample to the microwell and removal of same therefrom;
a first bore that accommodates a working electrode such that the working electrode is in contact with the contents of the microwell; and
a second bore that accommodates a reference electrode and a counter electrode, wherein the reference electrode and the counter electrode are in contact with the contents of the microwell and in proximity to the working electrode.

10. The apparatus of claim 9, wherein the working electrode comprises a graphite-containing sensor electrode comprising a nanocomposite comprising nitrate-binding metal ion complexes incorporated in and/or on a polyaniline/carbon (Pani/C) nanostructure.

11. The apparatus of claim 9, wherein the reference electrode and the counter electrode are positioned on the same electrode device.

12. The apparatus of claim 9, wherein first and second bores permit electrical connection of the working electrode and the reference and counter electrodes with an analytical device for sensing the presence of nitrate in the water sample.

13. The apparatus of claim 9, wherein liquid cannot escape from the microwell via the first and second bores comprising the respective electrodes.

14. The apparatus of claim 9, wherein the microfluidic cassette comprises a polymer or wood.

15. The apparatus of claim 9, wherein the inlet to, and the outlet from, the microwell are the same channel or separate channels.

16. The apparatus of claim 9, wherein the microfluidic cassette comprises wood, and the microwell, and the inlet and outlet has a waterproof/hydrophobic coating.

17. The apparatus of claim 9, wherein the microfluidic cassette comprises 1, 2, 3, 4, or 5 layers.

18. A method of measuring a concentration of nitrate ions in a water sample, said method comprising:
introducing a liquid buffer to the inlet of the apparatus of claim 9;
introducing the water sample to the apparatus by dipping the cassette in water or by introducing the water sample to the inlet of the cassette; and
measuring the impedance or electrical current using an electrochemical analyzer and converting same to nitrate concentration.

19. The method of claim 18, wherein the apparatus has a nitrate sensitivity of ΔR_{ct}≈2.3=0.089 Ωcm⁻² ppm⁻¹ for nitrate concentrations in a range from about 0.6 ppm to about 10 ppm and ΔR_{ct}≈0.36±0.043 Ωcm⁻² ppm⁻¹ for nitrate concentrations in a range from about 15 ppm to about 50 ppm.

20. The method of claim 18, wherein temperature is in the range of about 5° C. to about 65° C.
